(19) United States
(12) Patent Application Publication
(10) Pub. No.: US 2020/0171117 A1
(43) Pub. Date: Jun. 4, 2020

(54) DRY EYE BOTANICALS

(71) Applicant: ACCESS BUSINESS GROUP INTERNATIONAL LLC, Ada, MI (US)

(72) Inventors: Kelly M. GLYNN, Grand Rapids, MI (US); Kevin W. GELLENBECK, Poway, CA (US); Dawna Salter VENZON, Seal Beach, CA (US); Rohit P. DUGAR, Buena Park, CA (US); Mary A. MURRAY, Irvine, CA (US); Becky L. BENDER, Trabuco Canyon, CA (US)

(21) Appl. No.: 16/699,315

(22) Filed: Nov. 29, 2019

Related U.S. Application Data
(60) Provisional application No. 62/772,751, filed on Nov. 29, 2018.

(51) Int. Cl. A61K 36/9068; A61P 27/04; A61K 36/73; A61K 9/48

(52) U.S. Cl. CPC A61K 36/9068; A61K 9/48; A61K 36/73; A61P 27/04

(57) ABSTRACT

A composition for administration to a subject with dry eye or a dry eye-associated ocular condition is disclosed. The composition includes a botanical active agent in an amount effective for treating, preventing, and/or ameliorating dry eye or a dry eye-associated ocular condition in a subject upon administration of the composition thereto. The botanical active agent comprises a Zingiber officinale extract, an Eriobotrya japonica extract, or combinations thereof. The composition may be adapted for oral administration to the subject, and formulated as a nutraceutical, pharmaceutical, or supplement, e.g. in a single dosage form of a capsule or softgel. A method of treating, preventing, and/or ameliorating dry eye or a dry eye-associated ocular condition in a subject is also disclosed. The method comprises administering an effective amount of the composition to a subject.

### CROSS-REFERENCE TO RELATED APPLICATIONS

This application claims priority to U.S. Pat. Appl. No. 62/772,751, filed on 29 Nov. 2018, the content of which is incorporated by reference.

### FIELD OF THE INVENTION

The present invention relates generally to compositions and methods for treating ocular conditions and, more specifically, to a botanical extract-containing composition for treating dry eye or a dry eye-associated ocular condition in a subject and related methods.

### BACKGROUND OF THE INVENTION

Dry eye syndrome, which is also known as keratoconjunctivitis sicca and referred to more simply as “dry eye,” is a disease associated with dryness of the cornea and conjunctiva (i.e., the ocular surface) due to a lack of tears or excessive evaporation of tears. Such dryness of the ocular surface can lead to increased friction of the ocular surface and, in turn, inflammation and damage to the corneal epithelium, which is otherwise protected by the tear film. Dry eye may be further classified as aqueous tear-deficient dry eye syndrome, which is caused by the lack of tears secreted in lacrimal gland, or evaporative dry eye syndrome, which is caused by excessive moisture loss from the ocular surface due to the lack of a viscous substance in tears (e.g. occurring as a result of a reduced secretory ability of goblet cells of the conjunctiva and Meibomian gland of the eyelid). Factors that cause dry eye vary widely, and include, by way of example, inflammation of tear-producing organs, reduced tear secretion, excessive tear evaporation, certain systemic diseases such as Sjogren's syndrome or Stevens Johnson syndrome, and even prolonged and long-term use of various electronic devices with screens such as computers, smartphones, and tablets.

One potential cause of dry eye and, more particularly, evaporative dry eye syndrome (i.e., evaporative dry eye, or EDE), is atrophy of the Meibomian gland and eventual dysfunction in the aging eye. The Meibomian glands, also known as the tarsal glands, are holocrine-type exocrine glands located inside the tarsal plate along the rims of the eyelid, and are responsible for production of meibum (i.e., the main source of lipids for the human tear film). Dysfunction of the Meibomian glands leading to decreased or no production and/or secretion of lipids will prevent replenishment/formation of the natural barrier that is the tear film, leading to increased evaporation of the aqueous tear layer and the effects above, as well as leaving the ocular surface unprotected from microbial agents and environmental organic matter (e.g. dust, pollen, etc.).

Conventional treatments for dry eye are typically focused on addressing subjective symptoms of the patient. For example, conventional treatments include artificial tears and serum drops, with active agents such as hyaluronic acid, chondroitin sulfate, glutathione, and fibronectin. Unfortunately, however, therapeutic effects of such conventional treatments have not yet been sufficient. Moreover, such conventional treatment methods necessarily involve topical delivery, as well as frequent applications (i.e., daily or multiple times each day). Unsurprisingly, such conventional treatment methods directed at alleviating symptoms of dry eye are frequently reported as inconvenient and uncomfortable to use.

### BRIEF SUMMARY OF THE INVENTION

A composition for administration to a subject with dry eye or a dry eye-associated ocular condition is provided. The composition includes a botanical active agent in an amount effective for treating, preventing, and/or ameliorating dry eye or a dry eye-associated ocular condition in a subject upon administration of the composition thereto. The botanical active agent comprises a Zingiber officinale extract, an Eriobotrya japonica extract, or combinations thereof. The composition may be formulated as a nutraceutical, pharmaceutical, or supplement.

In some embodiments, the composition is formulated as an oral composition. The oral composition may be adapted for oral administration to a subject, direct oral consumption by a subject, or mixing with a foodstuff for subsequent consumption by a subject. In certain embodiments, the oral composition comprises a softgel or capsule shell encapsulating the botanical active agent, i.e., such that the oral composition is be formulated as a softgel or capsule. In particular embodiments, the composition is formulated as a topical composition. The topical composition may be adapted for topical administration to a subject, e.g. as an eye cream.

In some embodiments, the composition is formulated in a dosage form. In some such embodiments, the composition comprises an amount of the botanical active agent, per dose, effective to increase lipid synthesis in a Meibomian gland of a subject, increase meibum production of a subject, and/or increase meibum secretion of the subject, upon administration of the composition thereto. In these or other such embodiments, the composition comprises an amount of the botanical active agent, per dose, effective to increase expression of messenger ribonucleic acid (mRNA) in the subject for one or more of adipose differentiation-related protein (ADFP), very long chain fatty acids-like 4 (ELOVL4), and angiopoietin-like 4 (ANGPTL4), upon administration of the composition thereto. In these or other such embodiments, the composition comprises an amount of the botanical active agent, per dose, effective to activate peroxisome proliferator-activated receptor gamma (PPARγ) in Meibomian glands and/or inhibit nuclear factor kappa-light-chain-enhancer of activated B cells (NF-κβ) in cells of a subject, upon administration of the composition thereto.

In some embodiments, the composition further comprises an eye health additive component. In some such embodiments, the eye health additive component comprises lutein, beta carotene, lycopene, alpha carotene, zeaxanthin, docosahexaenoic acid (DHA), eicosapentaenoic acid (EPA), vitamin C, vitamin A, a bilberry extract, a citrus bioflavonoid extract, a black currant extract, a spinach dehydrate, or combinations thereof. The composition may also comprise a pharmaceutically acceptable additive.

A method of treating, preventing, and/or ameliorating dry eye or a dry eye-associated ocular condition in a subject is also provided. The method comprises administering an effective amount of the composition to a subject.

In certain embodiments, the dry eye-associated ocular condition comprises aqueous tear-deficient dry eye syndrome, evaporative dry eye syndrome, blepharitis (eyelid inflammation), keratoconjunctivitis sicca, eye redness, corneal ulcer due to dryness, or combinations thereof.

In some embodiments, the method includes administering the composition to the subject in an amount effective to increase lipid synthesis in a Meibomian gland of a subject, increase meibum production of a subject, and/or increase meibum secretion of the subject. In these or other embodiments, the method includes administering the composition to the subject in an amount effective to increase expression of messenger ribonucleic acid (mRNA) in the subject for adipose differentiation-related protein (ADFP), very long chain fatty acids-like 4 (ELOVL4), angiopoietin-like 4 (ANGPTL4), or combinations thereof. In these or other embodiments, the method includes administering the composition to the subject in an amount effective to activate peroxisome proliferator-activated receptor gamma (PPARγ) in Meibomian glands and/or inhibit nuclear factor kappa-light-chain-enhancer of activated B cells (NF-κβ) in cells of the subject.

These and other objects, advantages, and features of the invention will be more fully understood and appreciated by reference to the description of the current embodiments, examples, and drawings set forth herein.

Before the embodiments of the invention are explained in detail, it is to be understood that the invention is not limited to the details of operation or to the details of construction and the arrangement of the components set forth in the following description or illustrated in the drawings. The invention may be implemented in various other embodiments and practiced or carried out in alternative ways not expressly disclosed herein. Also, it is to be understood that the phraseology and terminology used herein are for the purpose of description and should not be regarded as limiting. The use of “including” and “comprising” and variations thereof is meant to encompass the items listed thereafter and equivalents thereof as well as additional items and equivalents thereof. Further, enumeration may be used in the description of various embodiments. Unless otherwise expressly stated, the use of enumeration should not be construed as limiting the invention to any specific order or number of components. Nor should the use of enumeration be construed as excluding from the scope of the invention any additional steps or components that might be combined with or into the enumerated steps or components.

### BRIEF DESCRIPTION OF THE DRAWINGS

FIG. 1 is a plot showing results of the response of a PPARγ regulated gene expression assay of an Eriobotrya japonica extract at varying concentrations;

FIG. 2 is another plot showing results of the response of a PPARγ regulated gene expression assay of an Eriobotrya japonica extract and major components thereof;

FIG. 3 is an additional plot showing results of the response of a PPARγ regulated gene expression assay of an Eriobotrya japonica extract and a PPARγ-specific antagonist; and

FIG. 4 is another plot showing results of the response of a PPARγ regulated gene expression assay of an Eriobotrya japonica extract, major components thereof, and a PPARγ-specific antagonist.

### DETAILED DESCRIPTION OF THE CURRENT EMBODIMENTS

A composition for administration to a subject with dry eye or a dry eye-associated ocular condition is provided herein. The composition comprises a botanical active agent comprising one or more particular botanical extracts. As will be understood in view of this disclosure, the composition is not particularly limited aside from the botanical active agent and, in particular, the extract(s) thereof, as well as the related components and methods. As such, the composition may be formulated as a nutraceutical, pharmaceutical, or supplement, and may be utilized as a unique and stand-alone therapeutic or in combination with other therapeutics compatible therewith.

The composition is useful for treating, preventing, and/or ameliorating dry eye or a dry eye-associated ocular condition. More specifically, as will be appreciated in view of the examples below, the composition of the present embodiments is believed to be capable of increasing, and/or preventing a decrease in, the expression of certain peroxisome proliferator-activated receptor gamma (PPARγ)-regulated response genes associated with desirable Meibomian gland functions. PPARγ is a nuclear receptor implicated in regulating adipocyte and sebocyte differentiation and lipogenesis, and is implicated in modulating Meibomian gland differentiation during aging. As such, the composition may be utilized to treat (i.e., slow, prevent, reverse, etc.) age-related Meibomian gland atrophy, which has been associated with decreased expression of PPARγ and, in turn, various dry eye factors including decreased meibocyte differentiation, lipid synthesis, and meibum production/secretion.

Additionally, the bioactivity of the botanical active agent provides the composition with improved utility in preventing and/or treating dry eye-associated ocular conditions from various environmental triggers, including atmospheric triggers (e.g. dry air, air-conditioning, heated air in cold climates, etc.), reading, night driving, prolonged/frequent use of digital screens for work or entertainment, etc. More specifically, the composition provides for improved ocular oil secretion (e.g. meibum), a decreased rate of water evaporation from the eye, decreased ocular inflammatory response, improved eye comfort, decreased eye irritation, and improved vision (e.g. via preventing fluctuations that may accompany episodes of dry eye).

As introduced above, the composition comprises a botanical active agent. More specifically, the botanical active agent comprises, optionally consists essentially of, or optionally consists of, at least one of a Zingiber officinale extract and an Eriobotrya japonica extract (collectively, the “botanical extracts”), which are each described in turn further below.

The term “extract” is used herein in the conventional sense to refer to a composition that has been obtained via fluid extraction from a source material. As such, the term “botanical extract” is to be understood as a composition obtained via fluid extraction (e.g. solvent extraction, gas extraction, CO2 extraction, etc.) from a botanical source (i.e., a plant material). Botanical extracts suitable for use in the composition can be obtained via any extraction method, or combination of such methods, known in the art, including water extractions, steam extractions, solvent extractions, etc. Exemplary extraction techniques are described below. However, the botanical extracts are not limited to a particular extraction method, or additional/adjuvant techniques used to obtain the botanical extracts, but rather may vary according to the parameters described herein. Additionally, an extraction step is not required to prepare the botanical active agent and/or the composition, as suitable extracts (e.g. standardized extracts) are readily available from a number of commercial suppliers.

Botanical extracts suitable for use in, or as, the botanical active agent include those obtained via solvent extraction, e.g. via use of a polar solvent such as an alcohol (e.g. methanol, ethanol, butylene glycol, etc.), ether (e.g. diethyl ether, methyl tert butyl ether, etc.), ketone (e.g. acetone), ester (e.g. ethyl acetate), phenol, water, and the like, a nonpolar solvent such as benzene, xylenes, toluene, etc., as well as derivatives, modifications, and combinations thereof (e.g. solvent-water blends, including alcohol-water, acetone-water, etc.). Additional and alternative extraction techniques include sequential fractionations, total hydro-ethanolic extractions, lump-sum extractions, supercritical fluid extractions (e.g. with CO2), and the like, as well as those utilizing sequential or secondary extractions from a first extract (e.g. a non-polar solvent extract of a botanical extract obtained from a polar solvent extraction) or other processing techniques such as filtration, purification, distillation, dehydration, evaporation, concentration, drying, etc. Specific examples of suitable extraction methods are described in U.S. Pat. No. 7,897,184, which is incorporated by reference herein.

In some embodiments, the botanical active agent, and thus the composition, comprises the Zingiber officinale extract, i.e., an extract comprising, optionally consisting essentially of material from the flowering plant Zingiber officinale. The Zingiber officinale extract is not particularly limited, and may comprise or be any extract or combination of extracts from a Zingiber officinale plant suitable for use in the embodiments herein. More specifically, exemplary Zingiber officinale extracts include those capable of activating PPARγ or eliciting/exhibiting any other such activities described herein as part of the botanical active agent.

Specific examples of Zingiber officinale extracts are known in art. As such, the Zingiber officinale extract may be purchased or otherwise obtained commercially from various sources, prepared (e.g. using any conventional extraction technique(s) known in the art, such as any of those described herein), or combinations thereof.

As will be understood by those of skill in the art, Zingiber officinale is primarily cultivated for its rhizome, which is commonly known as “ginger root” or, more simply, as “ginger,” which is widely used as a spice, flavorant, and component of certain nontraditional/folk medicines. Ginger comprises an oleoresin, which primarily comprises gingerol, shogaol, and, dehydrogingerdione. Ginger also typically comprises approximately 1-4 wt. % of volatile oils, including various shogaols, bisaboline, cineol, phelladrene, citral, borneal, citronellal, geramial, linalool, limonene, zingiberol, zingiberine, and camphene. Ginger also comprises phenolics such as zingerone, in addition to gingerol, as well as other components such as enzymes (e.g. zingibain), vitamins, minerals, fats, proteins, sugars, and fibers.

The Zingiber officinale extract may comprise material from any part of the plant, or combinations of parts, and is not limited to rhizomal extracts. For example, the Zingiber officinale extract may comprise material extracted from one or more parts of a Zingiber officinale plant, including the root, stem, rhizome, leaf, flower, seed, and/or fruit, thereof. Moreover, such extracts may be further processed (e.g. defatted, partially defatted, ground, dried, precipitated, washed, filtered, mesh-sorted, extracted, distilled, concentrated, etc.) to obtain the Zingiber officinale extract. Likewise, the Zingiber officinale plant may be extracted in raw form, or processed prior to extraction of the Zingiber officinale extract (e.g. used in raw form, suspended form, dehydrated form, concentrated form, etc.). As such, examples of extracts suitable for use in or as the Zingiber officinale extract typically comprise bisaboline, cineol, phelladrene, citral, boreal, citronellal, geramial, linalool, limonene, zingiberol, zingiberine, camphene, gingerol, shogaol, zingerone, zingibain, vitamin B6, vitamin C, calcium, magnesium, phosphorus, potassium, linoleic acid, pectins (i.e., pectic polysaccharidec, e.g. rhammose, arabinose, xylose, mannose, galactose, glucose, etc.), gallic acid, tannic acid, gentisic acid, protocatechuic acid, Vanillic acid, caffeic acid, syringic acid, cinnamic acid, and the like, as well as derivatives and combinations thereof. In certain embodiments, the botanical active agent comprises a Zingiber officinale extract comprising material obtained (i.e., extracted) from ginger root.

The amount of the Zingiber officinale extract utilized in the botanical active agent may vary, and will be selected based on the number and types of components being utilized in the botanical active agent. In certain embodiments, the botanical active agent comprises from 1 to 2000 mg of the Zingiber officinale extract, such as from 1 to 1000, optionally of from 2 to 500, or optionally of from 50 to 500, mg. However, amounts outside these ranges may also be utilized. For example, in certain embodiments, the botanical active agent includes the Zingiber officinale extract in an amount of at least 1, optionally of at about 50, optionally of at least 100, optionally of at least 250, optionally of at least 500, optionally of at least 1000, or optionally of at least 1500, mg. In these or other embodiments, an upper boundary may be selected such that the botanical active agent comprises the Zingiber officinale extract in an amount of ≤100, ≤250, ≤500, ≤750, ≤1000, ≤2000, ≤5000 mg. In various embodiments, the botanical active agent can include an amount of Zingiber officinale extract optionally in an amount of greater than 1, optionally greater than 5, optionally greater than 10, optionally greater than 25, optionally greater than 50, optionally greater than 75, optionally greater than 80, or optionally greater than 95, wt. %, based on the total weight of the botanical active agent. In such embodiments, an upper boundary may be selected to be generally ≤0, ≤20, ≤30, ≤40, ≤50, ≤60, ≤70, ≤80, ≤90, and ≤99 wt. %, respectively, based on the total weight of the botanical active agent.

In certain embodiments, the botanical active agent comprises more than one Zingiber officinale extract, such as 2, 3, 4, or more Zingiber officinale extracts. In such embodiments, each Zingiber officinale extract is independently selected, may be the same as or different from any other Zingiber officinale extract, and each utilized in an amount as described above.

The Zingiber officinale extract may be utilized in any form, such as neat (i.e., absent solvents, carrier vehicles, diluents, etc.), or disposed in a carrier vehicle, such as a solvent or dispersant. The carrier vehicle, if present, may comprise an aqueous solvent (e.g. water), an organic solvent, fluid, or oil, or the like, or combinations thereof. When utilized, the carrier vehicle will be selected based on the particular components of the botanical active agent and/or the composition, such as the particular Zingiber officinale extract(s) utilized. It will be appreciated that the Zingiber officinale extract may be combined with the carrier vehicle, if utilized, prior to, during, or after being combined with any other components of the botanical active agent and/or composition.

In some embodiments, the botanical active agent, and thus the composition, comprises the Eriobotrya japonica extract, i.e., an extract comprising, or optionally consisting essentially of material from the flowering plant Eriobotrya japonica , which is commonly referred to as a loquat tree. As will be understood by those of skill in the art, Eriobotrya japonica is primarily cultivated for loquat fruits and/or as a decorative tree or shrub.

The Eriobotrya japonica extract is not particularly limited, and may comprise or be any extract or combination of extracts from an Eriobotrya japonica plant suitable for use in the embodiments herein. More specifically, exemplary Eriobotrya japonica extracts include those capable of activating PPARγ or eliciting/exhibiting any other such activities described herein as part of the botanical active agent. Specific examples of Eriobotrya japonica extracts are known in art. As such, the Eriobotrya japonica extract may be purchased or otherwise obtained commercially from various sources, prepared (e.g. using any conventional extraction technique(s) known in the art, such as any of those described herein), or combinations thereof.

The Eriobotrya japonica extract may comprise material from any part or combinations of parts of the plant. For example, the Eriobotrya japonica extract may comprise material extracted from one or more parts of an Eriobotrya japonica plant, including the root, stem, leaf, flower, seed, and/or fruit, thereof. Moreover, such extracts may be further processed (e.g. defatted, partially defatted, ground, dried, precipitated, washed, filtered, mesh-sorted, extracted, distilled, concentrated, etc.) to obtain the Eriobotrya japonica extract. Likewise, the Eriobotrya japonica plant may be extracted in raw form, or processed prior to extraction of the Eriobotrya japonica extract (e.g. used in raw form, suspended form, dehydrated form, concentrated form, etc.).

In certain embodiments, the botanical active agent comprises an Eriobotrya japonica extract comprising material obtained (i.e., extracted) from Eriobotrya japonica leaves. As such, in some embodiments the Eriobotrya japonica extract comprises one or more triterpenoids, such as tormentic acid, euscaphic acid, ursolic acid, as well as derivatives and/or precursors thereof (e.g. methoxylated and/or polyhydroxylated variants of such acids and related tepenoids), and combinations of such compounds. In these or other embodiments, the Eriobotrya japonica extract comprises at least one, optionally at least two, or optionally at least three of maslinic acid, oleanolic acid, tormentic acid, euscaphic acid, ursolic acid, and cosolic acid.

The amount of the Eriobotrya japonica extract utilized in the botanical active agent may vary, and will be selected based on the number and types of components being utilized in the botanical active agent. In certain embodiments, the botanical active agent comprises from 1 to 2000 mg of the Eriobotrya japonica extract, such as from 1 to 1000, optionally of from 2 to 500, or optionally of from 50 to 500, mg. However, amounts outside these ranges may also be utilized. For example, in certain embodiments, the botanical active agent includes the Eriobotrya japonica extract in an amount of at least 1, optionally of at about 50, optionally of at least 100, optionally of at least 250, optionally of at least 500, optionally of at least 1000, or optionally of at least 1500, mg. In these or other embodiments, an upper boundary may be selected such that the botanical active agent comprises the Eriobotrya japonica extract in an amount of ≤100, ≤250, ≤500, ≤750, ≤1000, ≤2000, ≤5000 mg. In various embodiments, the botanical active agent can include an amount of Eriobotrya japonica extract optionally in an amount of greater than 1, optionally greater than 5, optionally greater than 10, optionally greater than 25, optionally greater than 50, optionally greater than 75, optionally greater than 80, or optionally greater than 95, wt. %, based on the total weight of the botanical active agent. In such embodiments, an upper boundary may be selected to be generally ≤0, ≤20, ≤30, ≤40, ≤50, ≤60, ≤70, ≤80, ≤90, and ≤99 wt. %, respectively, based on the total weight of the botanical active agent.

In certain embodiments, the botanical active agent comprises more than one Eriobotrya japonica extract, such as 2, 3, 4, or more Eriobotrya japonica extracts. In such embodiments, each Eriobotrya japonica extract is independently selected, may be the same as or different from any other Eriobotrya japonica extract, and each utilized in an amount as described above.

The Eriobotrya japonica extract may be utilized in any form, such as neat (i.e., absent solvents, carrier vehicles, diluents, etc.), or disposed in a carrier vehicle, such as a solvent or dispersant. The carrier vehicle, if present, may comprise an aqueous solvent (e.g. water), an organic solvent, fluid, or oil, or the like, or combinations thereof. When utilized, the carrier vehicle will be selected based on the particular components of the botanical active agent and/or the composition, such as the particular Eriobotrya japonica extract(s) utilized. It will be appreciated that the Eriobotrya japonica extract may be combined with the carrier vehicle, if utilized, prior to, during, or after being combined with any other components of the botanical active agent and/or composition.

In some embodiments, the botanical active agent comprises both the Zingiber officinale as well as the Eriobotrya japonica extract. In such embodiments, the botanical extracts may be included in the botanical active agent in a ratio (e.g. wt./wt.) relative to the other extract(s). For example, the botanical active agent may comprise a mass ratio of the Zingiber officinale extract to the Eriobotrya japonica extract of from 1000:1 to 1:1000, such as from 100:1 to 1:100, optionally of from 10:1 to 1:10, optionally of from 5:1 to 1:5, optionally of from 4:1 to 1:4, optionally of from 3:1 to 1:3, optionally of from 2:1 to 1:2, optionally of from 1.5:1 to 1:1.5, or optionally of 1:1. However, ratios outside and in between these ranges may also be utilized, such as from 1:10 to 1:100, optionally of from 1:10 to 1:1000, optionally of from 10:1 to 100:1; optionally of from 1000:1 to 10:1; optionally of from 1:2 to 1:4; optionally of from 4:1 to 2:1; optionally of from 1:3 to 1:5; optionally of from 5:1 to 3:1; optionally of from 100:1 to 1:100; or optionally of from 1:1 to 2:1.

The composition may include any amount of the botanical active agent, which will be selected based on the number and types of components being utilized in the composition as a whole. In certain embodiments, composition comprises the botanical active agent in an amount of from 1 to 2000 mg, such as from 5 to 1750, optionally of from 10 to 1500, optionally of from 15 to 1250, optionally of from 20 to 1000, optionally of from 25 to 750, optionally of from 30 to 500, optionally of from 35 to 500, optionally of from 40 to 500, optionally of from 45 to 450, optionally of from 50 to 450, or optionally of from 50 to 400 mg. However, amounts outside and/or overlapping with these ranges may also be utilized. For example, it is to be appreciated that the ranges described above with respect to the amount of each botanical extract in the botanical active agent may equally apply to the amount of each botanical extract in the composition as a whole, such as when the botanical active agent consists of but one of botanical extracts.

In general, the composition is not limited in terms of formulation, peripheral ingredients, form, number of functions, etc., aside from comprising the botanical active agent and the botanical extract(s) thereof. Rather, the composition may be varied, and may be formulated in any fashion consistent with this disclosure.

As introduced above, and described in additional detail below, the composition is adapted for ameliorating a condition of and/or confer a health benefit to a subject. As such, the composition comprises an active agent (i.e., a compound or composition capable of eliciting a particular biological effect in a subject). Typically, the active agent comprises, optionally is, the botanical active agent (i.e., comprises, optionally consists essentially of, or optionally consists of, the botanical extract(s) thereof). However, in various embodiments, other active agents (e.g. probiotics, prebiotics, parabiotics, pharmaceuticals, nutraceuticals, anesthetics, counterirritants, chondroprotective agents, etc.) may be utilized in the composition in addition to the botanical active agent. For example, in certain embodiments, the composition comprises a pharmaceutically acceptable additive, which may comprise or be one or more of the various components described below. In other embodiments, however, the composition is substantially free from, or optionally free from other active agents (i.e., other than the botanical active agent and the botanical extract(s) thereof).

In particular embodiments, the composition comprises a combination of the botanical active agent and the additional active agent (e.g. eye health additive). In such embodiments, the composition may be homogeneous or mixed as a unitary composition or, alternatively, may be adapted as a kit including a first component comprising the botanical active agent and a second component comprising the additional active agent. In some such embodiments, the additional active agent is a pharmaceutical agent (e.g. an angiotensin converting enzyme (ACE) inhibitor, an anti-inflammatory agent, a vasodilator, an immune modulating agent (i.e., an antibody therapeutic agent), an analgesic, an antibiotic, etc.). The components of the kit may be administered together or separately (e.g. sequentially, in any order).

In addition to the components described above (i.e., the botanical active agent, the pharmaceutically acceptable additive, etc.), the composition may comprise any number of additional ingredients/components. For example, in some embodiments, the composition comprises an additive component, which may comprise one or more additives. Examples of suitable additives for use in the additive component include amino acids, peptides, proteins, lipids, vitamins, carbohydrates, nucleic acids, minerals, nutrients, antioxidants, probiotic bacterial strains, lipotropic agents, extracts, concentrates, oils, gums, and combinations thereof.

In certain embodiments, the additive component comprises a flavoring agent, a dye, a flow modifier, a preservative, a filler, a binder, a dispersing agent, a solubilizer, a supplemental nutrient, an excipient, a buffer, a lubricant, a sweetener, a wetting agent, or any combination thereof. Particular examples of suitable additives include vitamin A, vitamin D, calcium, methyl cellulose, lecithin, lysolecithin, and long-chain fatty alcohols. In particular embodiments, the additive component comprises a carrier, such as a consumable, nutritional, and/or pharmaceutical carrier, or a combination thereof.

In certain embodiments, the additive component comprises one or more of the following: excipients, such as diluents and binders; granulating agents; glidants (or flow aids); fillers; lubricants; stabilizers; bulking agents; anti-caking agents; coatings; disintegrants; fragrances; natural or artificial sweeteners; flavorings; and pigments; alcohols, such as ethanol, propyl alcohol and benzyl alcohol; glycerin; glyceryl triacetate; mineral oils; water; silicones, such as silicone oils; silicon dioxide; waxes, such as carnauba wax and beeswax; fatty esters and fatty alcohols; carob; corn syrups, such as hydrolyzed corn syrup solids; cellulose, such as methyl cellulose, hydroxypropyl methyl cellulose, carboxy methyl cellulose, microcrystalline cellulose, and powdered cellulose; fructose; maltodextrin and maltol, such as natural maltol; sorbitol; preservatives, such as p-hydrobenzoic acid esters; potassium sorbate; sodium benzoate; flow agents; stearates, such as calcium stearate, magnesium stearate, and sodium magnesium stearate; dicalcium phosphate; vegetable oils, such as hydrogenated vegetable oils; antioxidants, such as ascorbic acid or tocopherol; starches, such as corn starch and potato starch; glycols and polyglycols; moisturizers; emollients; emulsifiers; surfactants; oils; extracts; skin protectants; disinfectants; antiseptics; drugs and drug substances; analgesic compounds; anti-neuralgic compound; anti-oxidants; blood circulation promoters; antidepressant compounds; anti-anxiety compounds; anti-stress compounds; colorants; fillers; solvents; vehicles; carriers; other types of additives known to those of skill in the art (e.g. nutraceutical additives); and combinations thereof.

In particular embodiments, the additive component comprises an eye health additive. In such embodiments, the additive component may be further defined or otherwise referred to as an eye health additive component, and comprises, optionally consist essentially of, or optionally consists of an eye health additive (i.e., a compound or composition providing one or more eye functions and/or structures. For example, the eye health additive component may be adapted to provide to a subject, upon administration of the composition thereto, antioxidant support (e.g. to protect the central retina from oxidative damage, protect the retina from damage from blue light energy from digital/electronic devices, protect the ciliary body of the eye to help with normal lens focus such in situations of visual strain or fatigue, to maintain/manage lens health and ocular pressure within the eye, etc.), support for forming visual pigments and improving macular pigment density (e.g. to facilitate good visual performance), support for normal visual adaptation in response to changes in light, and other such supportive and/or beneficial effects. Particular examples of such eye health additives include lutein, beta carotene, lycopene, alpha carotene, zeaxanthin, docosahexaenoic acid (DHA), eicosapentaenoic acid (EPA), vitamin C, vitamin A, a bilberry extract, a citrus bioflavonoid extract, a black currant extract, a spinach dehydrate, and the like, as well as derivatives, modifications, and combinations thereof. In certain embodiments, the eye health additive component comprises one or more of the following: vitamins, nutritional supplements, and/or minerals known in the art to be generally beneficial to eye health, including Vitamin E, Vitamin C, beta-carotene (Vitamin A), zinc, lutein, copper, taurine, docosahexaenoic acid (DHA), eicosapentaenoic acid (EPA), and combinations thereof.

Of course, components aside from the additive component may also be utilized in the composition. For example, the composition may comprise a fat component, a lipid component, a protein component, a fiber component, a carbohydrate component, and the like, or combinations thereof, which may be independently selected, e.g. based on the desired formulation, form, and/or end use of the composition, as will be understood by those of skill in the art in view of the description herein. For example, as described further below, the composition may be formulated for oral administration to a subject. In such embodiments, the composition may comprise an effective amount of one or more sweeteners, including carbohydrate sweeteners and natural and/or artificial no/low calorie sweeteners, selected based on the type and/or intensity of sweetness intensity desired.

In certain embodiments, the composition comprises a vehicle (i.e., a carrier vehicle, carrier, etc.) that may be independently selected (e.g. based on the desired formulation, form, and/or end use of the composition, etc.). Suitable vehicles and vehicle components are well known in the supplement, cosmetic, and pharmaceutical arts, and include water (e.g. purified, deionized, etc.); organic solvents such as alcohols, glycols (e.g. propylene glycol, pentylene glycol, butylene glycol, glycerol/glycerin, etc.), aliphatic alcohols (e.g. lanolin); mixtures of water and organic solvents (such as water and alcohol), and mixtures of organic solvents such as alcohol and glycerol (optionally also with water); lipid-based materials such as fatty acids, acylglycerols (e.g. oils, such as mineral oil, and fats of natural or synthetic origin), phosphoglycerides, sphingolipids, and waxes; protein-based materials such as collagen and gelatin; silicone-based materials (both non-volatile and volatile) such as cyclomethicone, dimethiconol, and dimethicone copolyol; hydrocarbon-based materials such as petrolatum, hydrogenated polyisobutene, and squalane; emollient esters (such as diisobutyl adipate and caprylates), thickening agents (acrylates (carbomers), acrylamides, acryl tau rates, hydroxyethylcellulose, methyl cellulose, xanthan gum, etc.), and the like, as well as derivatives, modifications, and combinations thereof. In some embodiments, the composition a cosmetically or pharmaceutically acceptable vehicle, such as saline, buffered saline, 5% dextrose in water, borate-buffered saline containing trace metals, and the like, any of those described herein, and combinations thereof.

It is to be appreciated that each additive may be utilized in the composition in any amount, which is typically selected based on the type of additive, the formulation of the composition, a desired end use of the composition, etc. It is also to be appreciated that certain additives may be classified under different terms of art, and may have similar, overlapping, or different functions with additives having different classifications. In certain embodiments, each additive is present in the composition in an amount of from greater than 0 to 75 wt. %, based on the total mass of the composition, such as in an amount of 1, 2, 3, 4, 5, 10, 15, 20, 25, 30, 35, 40, 45, 50, 55, 60, 65, 70, or 75 wt. %, based on the total mass of the composition, or in an amount in a range bounded by any two of such values.

Certain embodiments of the composition, which vary in terms of formulation and/or form, are described below. However, as introduced above, the composition is not particularly limited with regard to substance, form, number of functions, etc. and may comprise any number of components/ingredients in addition to the botanical active agent, such as the active agents and/or additives described above. In general, the components of composition will be individually or collectively selected based on an intended use and/or form of the composition, as will be readily understood by those of skill in the art.

Typically, the composition is formulated or otherwise adapted for administration to a mammalian subject (e.g. a human). For example, in various embodiments, the composition is adapted to be consumed and/or orally administered to a human subject. As such, the particular additives, carriers, adjuvants, fillers, etc. present in or combined with the composition may vary. Moreover, the physical form of the composition is not limited, and will be selected based on the particular components of the composition, a desired use of the composition, etc. As such, as will be understood in view of the description herein, the composition may be formulated as a liquid, dry powder, suspension, emulsion, gel, paste, etc., and combinations thereof. In certain embodiments, the composition is formulated as a sterile, non-pyrogenic liquid solution or suspension, a coated capsule, a suppository, a lyophilized powder, a transdermal patch, a softgel, or other forms are known. Other examples of suitable forms include solids, gels, liquids, creams, lotions, pomades, mousses, powders, foams, sprays, ointments, or other such preparations where the botanical active agent is disposed in an appropriate carrier vehicle, such as any of those described herein. In particular embodiments, the composition is formulated or otherwise provided as an eye cream.

In some embodiments, the composition is formulated as a nutraceutical composition, i.e., is adapted for use as a dietary supplement, food additive, food ingredient, or supplemented food product, and the like. For example, in certain embodiments, the composition comprises a softgel or capsule shell encapsulating the botanical active agent and, optionally, other components of the composition (e.g. the eye health additive component, etc.). In such a form, the in vivo release profile of the botanical extracts from may be a conventional (i.e., unmodified) release, a controlled release/sustained release (CR/SR), a time release, a targeted release, or an extended release. The particular release profile may generally be tailored be varying the encapsulant and/or other components of the composition, e.g. based on desired matrix erosion, changes in gel thickness, electrolyte ionization, and ionic interaction mechanisms. Common time and/or controlled release delivery systems include, but are not be limited to, starches, osmotic pumps, or gelatin micro capsules. Other examples of such nutraceutical forms of the composition are described below.

In certain embodiments, the composition is adapted to be mixed with a foodstuff or beverage. The term “foodstuff” is used herein to refer to a material that may be used as a food. As such, in certain instances the term foodstuff is used to describe a composition that may be consumed (e.g. by eating) by a living organism (e.g. a mammal), for nourishment and/or sustenance. Likewise, the term “beverage” as used herein refers to a potable liquid or other non-solid composition. However, in certain instances, the term beverage is used to describe a non-solid (e.g. liquid, slurry, suspension, etc.) composition that may be consumed by a living organism for nourishment and/or sustenance. As such, in particular instances the terms “beverage” and “foodstuff” may overlap. In certain instances, the term “nutritional composition” is used to describe a foodstuff and/or beverage formulation that can be eaten or drunk by a human subject for nutrition. Accordingly, in some embodiments, the composition is, optionally is a component of, a foodstuff or beverage. In these or other embodiments, the composition may be further defined as a food additive. As used herein, the term “food additive” refers to an ingredient, additive, component, or supplement suitable for incorporation in a foodstuff and/or beverage to confer a technical, nutritional, and/or health benefit (i.e., a function) to a host that consumes the foodstuff and/or beverage. Accordingly, such benefits may be closely related to the presence of the botanical active agent in the composition. The food additive can be added to different types of food including, but not limited to, medical foods, dietetic foods, and supplements. Certain aspects of the present embodiments can include the use of the composition as a food additive, as well as the use of the composition in methods of preparing foodstuffs and/or beverages.

In general, when utilized as a component of a foodstuff or beverage, the foodstuff or beverage comprises an admixture of the composition with one or more feed products, liquids, supplements, or combinations thereof. However, in certain embodiments, the composition may itself be further defined as a foodstuff or beverage composition, depending on the quantity, nature, and identity of individual additives and components present in the composition, such as those described herein. Thus, it is to be appreciated that the embodiments described herein with respect to the composition in general may equally encompass the foodstuff or beverage, a food or beverage product, and/or a food supplement comprising the composition. Accordingly, any amounts and/or examples of such components (e.g. in addition to the botanical active agent) described herein with respect to the composition itself may equally apply to the foodstuff or beverage comprising the composition, as will be understood by one of skill in the art.

In some embodiments, the foodstuff or beverage comprising the composition is further defined as a nutritional composition. In these or other embodiments, the nutritional composition is in the form of a dry food concentrate, which may be mixed with liquid or food and subsequently consumed. In some embodiments, the foodstuff or beverage comprising the composition is further defined as a medical food. In such embodiments, the medical food comprises the composition, and may be the same as or different from the nutritional composition described above. As used herein, the term “medical food” is typically used to refer to a food for a special dietary use, such as a food formulated for dietary management of a medical condition (e.g. based upon scientific or medical evaluation). However, it is to be appreciated that the term “medical food” may have one or more particular definitions depending on, for example, geographic location, specific use, regulatory agency, and the like. For example, in certain cases, the term medical food may be defined as a food which is formulated to be consumed or administered enterally under the supervision of a physician and which is intended for the specific dietary management of a disease or condition for which distinctive nutritional requirements, based on recognized scientific principles, are established by medical evaluation (see, e.g. section 5(b) of the Orphan Drug Act (21 U.S.C. 360ee (b) (3)), which is incorporated herein by reference). In these or other instances, the term medical food may be defined as a food for special dietary use as a food that has been specially processed or formulated to meet the particular requirements of a person: (a) in whom a physical or physiological condition exists as a result of a disease, disorder, or injury; or (b) for whom a particular effect, including but not limited to weight loss, is to be obtained by a controlled intake of food (see, e.g. section 6.24.001 of the Canadian Food and Drug Regulations (FDR, C.R.C., c. 870)(as amended 13 Jun. 2017)), which is incorporated herein by reference).

In certain embodiments, the composition may be further defined as a nutritional supplement, or as a complete nutritive. As used herein, the term “supplement” relates to a nutritional supplement which is a concentrated source of nutrient or optionally other substances with a nutritional or physiological effect whose purpose is to supplement the normal diet. For example, the composition may be formulated to provide a subject (e.g. a human), via consumption of the composition, with at least 1%, optionally at least 2%, optionally at least 5%, optionally at least 10%, optionally at least 25%, optionally at least 50%, of daily calories required by the subject. In particular embodiments, the composition may be formulated to provide the subject, via consumption of the composition, with 0.1 to 15, optionally from 0.2 to 10% of daily calories required by the mammal. However, it is to be appreciated that a daily calorie requirement is dependent on several factors, including the gender, height, and/or age of the subject, and thus the percentage of caloric requirement provided by the composition will be dependent on the particular person consuming the nutritional composition composed therefrom. For example, a 30 year old human male of 80 kg body weight and 180 cm height has a daily calorie requirement of around 2900 cal (calories) to maintain his body weight whereas a 30 year old human female of 55 kg body weight and 165 cm height has a daily calorie requirement of around 2100 cal to maintain her body weight. Moreover, daily caloric requirements may be reduced from the amount required to maintain body weight, e.g. where the mammal is intentionally reducing body weight.

The composition may be formulated to systemic or local administration. For systemic use, the composition is typically formulated for parenteral (e.g., intravenous, subcutaneous, intramuscular, intraperitoneal, intranasal or transdermal) or enteral (e.g., oral or rectal) delivery according to conventional methods. Intravenous administration can be by a series of injections or by continuous infusion over an extended period. Administration by injection or other routes of discretely spaced administration can be performed at intervals ranging from weekly to once to three times daily. Alternatively, the composition may be administered in a cyclical manner (administration of disclosed composition; followed by no administration; followed by administration of disclosed composition; and the like). Treatment can continue until the desired outcome is achieved. Alternatively, administration of the compositions may be continual, and thereby be a preventative administration, rather than an administration for treatment. It is to be appreciated, however, that the composition, e.g. itself and/or when formulated as described in any of the embodiments above (e.g. as the nutritional composition) is distinguished from a vaccine. In particular, each of the various compositions described herein may be free from, optionally substantially free from, a vaccine.

The composition may be presented in a unit dosage form, such as in a pack (e.g. metal or plastic foil, blister pack, etc.) or dispenser device including one or more unit doses of the composition. The pack or dispenser device may be accompanied by instructions for administration. Parenteral preparations may comprise sterile or sterilized products. Other useful dosage forms can be prepared by methods and techniques that will be well understood by those of skill in the art and may include the use of additional ingredients in producing tablets, capsules, or liquid dosage forms. Although exemplary dosages, dose frequencies, and methods of administration are discussed herein, these are merely exemplary and it is to be understood that the dose, dose frequency, and mode of administration may vary according to the age, body weight, condition and response of the individual consumer or patient, and the particular formulation of the composition.

Regardless of the particular form and/or formulation, including each of the compositions and forms described relative to the embodiments above, the composition typically comprises the botanical active agent in an amount sufficient to provide from 1 mg to 2 g of the botanical extracts (i.e., the Zingiber officinale and/or the Eriobotrya japonica extract), e.g. per dose and/or serving, or in total. For example, in certain embodiments, the composition is formulated to provide the botanical extracts in an amount of from 10 mg to 1g per dose, such as from 10 to 750, optionally from 10 to 500, optionally from 20 to 500, optionally from 30 to 500, optionally from 50 to 500, optionally from 50 to 250, optionally from 50 to 100 mg, per dose of the composition. In particular embodiments, the composition is formulated to provide the botanical extracts in an amount of from 10 mg to 3 g per dose, such as from 50 to 2500, optionally from 50 to 2250, optionally from 100 to 2000, optionally from 150 to 2000, optionally from 200 to 2000, optionally from 250 to 2000, optionally from 300 to 2000, optionally from 350 to 2000, optionally from 400 to 2000, optionally from 450 to 2000, or optionally from 500 to 2000, mg, per dose of the composition. In such embodiments, the dose may be a single dose or, optionally, may be defined as a daily dose (i.e., where multiple doses are taken, such as multiple doses per day, multiple single-daily doses, etc.).

It is to be appreciated that the composition may comprise multiple doses of the botanical extracts, such as in the amounts described above, and thus may comprise any amount of the botanical extracts in total (e.g. such as an amount greater than 500 g, optionally greater than 1, 2, 5, 10, 50, 100, 500, or even 1000 kg). Likewise, as described above, the composition may comprise components other than the botanical extracts. As such, it will be appreciated that the composition may comprise the botanical extracts in various concentrations, such as from 0.001 to 99 wt. % based on the total weight of the composition (i.e., wt./wt.). For example, in particular embodiments, the composition comprises the botanical extracts in an amount of from 1 to 98, optionally from 1 to 95, optionally from 1 to 90, optionally from 1 to 80, optionally from 1 to 70, optionally from 1 to 60, optionally from 1 to 50, optionally from 5 to 50, optionally from 10 to 50, optionally from 20 to 50 wt. % based on the total weight of the composition. In certain embodiments, the composition comprises the botanical extracts in an amount of from 0.001 to 10, optionally from 0.001 to 7.5, optionally from 0.001 to 5, optionally from 0.001 to 2.5, optionally from 0.001 to 1, optionally from 0.005 to 1, optionally from 0.01 to 1 wt. % based on the total weight of the composition. It is to be appreciated that the amounts above may be descriptive of the botanical extracts collectively (i.e., the amount of the botanical active agent present in the composition), or may be applied to each individual botanical extract (i.e., such that the Zingiber officinale extract and/or the Eriobotrya japonica extract are individually present in such a concentration.

In various embodiments, the amount of the botanical extracts is selected so as to activate PPARγ (i.e., upregulate the expression of PPARγ or otherwise induce an increase in PPARγ activity within a cell, e.g. relative to a cell not administered with the composition), upon administration of the composition to a subject. In certain embodiments, the botanical active agent and the botanical extract(s) thereof is present in the composition in an amount effective for improving the function of Meibomian glands and the lipid synthesis and secretion thereby of a subject administered the composition. In these or other embodiments, the botanical active agent is present in an amount effective for preventing evaporation of an aqueous tear film of an eye of a subject administered the composition. In these or other embodiments, the botanical active agent is present in an amount effective to inhibit NF-κB (i.e., effecting the down-regulation or otherwise reducing activity of NF-κB in a cell, e.g. relative to a cell not exposed to the composition), upon administration of the composition to a subject. In various embodiments, the botanical active agent and the botanical extract(s) thereof is present in an amount effective to have anti-inflammatory benefits for eye health of a subject administered the composition. These and various other physiological criterion may be used as motivation to select among the various compositional ranges described above below. For example, the amount of the botanical active agent and/or one or more of the botanical extracts thereof may be so selected as to effect a desired change in gene expression, metabolic activity (e.g. lipid synthesis), protein activity, transcriptional activity, or mRNA levels in affected cells of a subject upon administration of the composition.

As described above, the composition may be formulated in various ways to facilitate administration of the same to a subject (e.g. via consumption, oral administration, etc.). More specifically, the botanical extracts of the botanical active agent of the composition may be administered to the subject to confer a benefit thereto, as described in further detail below. Accordingly, a method of utilizing the composition (the “treatment method”) is provided, and is useful in ameliorating a condition of the subject. In general, the treatment method comprises administering the composition to the subject.

The composition may provide or mediate a particular therapeutic and/or prophylactic effect, and thus may be used (e.g. according to the treatment method) to treat or ameliorate a condition in a subject. As used herein, the term “treat” refers to an approach for obtaining beneficial or desired results including a therapeutic benefit and/or a prophylactic benefit. A therapeutic benefit can be partial or complete eradication or amelioration of an underlying disorder being treated, whether temporarily or permanently. As such, a therapeutic benefit can be achieved with the eradication or amelioration of one or more physiological symptoms associated with the underlying disorder, such that an improvement is observed in the subject, notwithstanding that the subject may still be afflicted with the underlying disorder. A prophylactic effect includes delaying, preventing, and/or eliminating the onset and/or appearance of a disease or condition, slowing, halting, and/or reversing the progression of a disease or condition, or combinations thereof. As such, any of these effects may be a prophylactic benefit, and may be achieved in a subject at risk of developing a particular disease. Accordingly, a subject reporting one or more physiological symptoms of a disease may undergo treatment (e.g. with the composition), even in the absence of a diagnosis of the disease.

The subject is not limited. Typically, however, the subject is a mammal (i.e., vertebrates of the class Mammalia, such as dogs, cats, goats, sheep, pigs, cattle, horses, donkeys, camels, and the like) and, more specifically, is a human (which may be referred to as “people” and/or “person(s)” herein). When describing a human, the term “adult” is typically used herein to refer to a human that has reached sexual maturity. By contrast, the terms “child” and “juvenile” are used herein to refer to a human that has not yet reached sexual maturity. Typically, the term “child” means a human subject between the stage of birth and the age of about 10 (i.e., childhood), and the term “juvenile” means a human subject that is greater than the age of about 10 and who has not completed the stage of puberty. Of course, the terms child, juvenile, adult, and infant are all encompassed by the term “human”, which is itself a subcategory of mammal as defined herein.

The composition may be administered to the subject in any form. Typically, the composition is administered orally and/or topically (e.g. as the consumable and/or topical formulations described above). However, other routes of administration may also be utilized.

When formulated for oral administration, the composition may be presented in discrete units (e.g. softgels, capsules, cachets, lozenges, tablets, etc.), each containing a predetermined amount of the composition (e.g. a recommended dose). For example, such unit doses may be utilized when the composition is formulated as the nutraceutical, or as the food additive and/or nutritional supplement. However, the composition may compose any form, such as a dry powder, a solution, a suspension, an emulsion, or the like, which may be presented in bulk form. For example, in certain embodiments, composition is formulated as the dry powder. In such embodiments, the treatment method may comprise measuring a dose of the composition to be administered, mixing the dose with foodstuff to form the food or beverage comprising the composition, and orally administering the food or beverage to the subject. In certain embodiments, as described above, the composition is adapted to be consumed as a liquid. In such embodiments, the treatment method may include combining a dose of the dry powder with a consumable liquid (e.g. water, juice, etc.) to form a consumable liquid (e.g. solution, suspension, emulsion, etc.) comprising the composition, and orally administering the consumable liquid (i.e., beverage) to the subject. In these or other embodiments, the dose of the composition to be administered may be pre-mixed with the foodstuff to form a food or beverage product comprising the composition, which may be subsequently consumed by the subject (e.g. via assisted, supervised, or self-administration).

When formulated for topical administration (i.e., as the topical composition), the composition may be presented in discrete units (e.g. pouches, packets, pods, etc.), each containing a predetermined amount of the composition (e.g. a recommended dose). For example, such unit doses may be utilized when the topical composition is formulated as the ointment, paste, cream (e.g. eye cream), lotion, or gel, described above. Additionally, such unit doses may be presented as a patch, i.e., with a unit dose of the topical composition in one of the forms above disposed on a backing member, which is adapted to support and secure the composition to a surface of the subject. The topical composition may also be presented in bulk form, such that the treatment method may comprise measuring a dose of the topical composition to be administered before administration. Examples of bulk forms include multi-dose amounts of the topical composition combined in a single container (e.g. a tub, jar, tube, can, etc.). Such containers may comprise a dispenser, such as in the case of a pump jar, lotion dispenser, pump spray jar, etc., such that measuring the dose of the topical composition is made convenient to the subject or a person administering the topical composition thereto.

The composition, in any form, may be administered as needed, daily, several times per day or in any suitable regimen such that a desired outcome is achieved. In the treatment method, the frequency of administration can depend on several factors, including a desired level of prevention or amelioration. Generally, an exemplary regimen includes administration of the composition to the subject once or twice daily, e.g. including an administration in the morning and/or an administration in the evening. The amount of the composition administered to the subject during each administration (i.e., the dose) may depend on several factors, such as the level of results desired, and the specific composition being utilized, the number of doses being administered, etc. In general, the composition is administered in a therapeutically or physiologically effective amount. As used herein, the term “therapeutically effective amount” relates to an amount (i.e., a quantity) of a composition (e.g. the composition of the present embodiments) required to achieve a particular therapeutic and/or prophylactic effect, such as in treating a subject (e.g. by ameliorating a condition thereof). Likewise, as used herein, the term “physiologically effective amount” relates to an amount of a composition (e.g. the composition of the present embodiments) required to achieve a desired physiological effect. Such effective amounts are typically measured and/or expressed in terms of the amount of the composition over time (e.g. g/day, mg/day, etc.), but may also incorporate the body weight of the subject (e.g. in kg), as expressed by the unit g/kg/day. Typically, the composition is administered in an amount effective to provide the botanical extracts of the botanical active agent to the subject. In certain embodiments, the composition is administered in an amount effective to ameliorate a condition of the subject. In these or other embodiments, the composition is administered in an amount effective to ameliorate at least two conditions of the subject, including any of those described herein.

As described above, the treatment method may be used to ameliorate a condition of the subject, such as one or more of those described above, by mediating a biological effect in the subject. For example, the treatment method may be used to ameliorate a condition affecting a subject's eye health, such as dry eye or a dry eye-associated ocular condition. In certain embodiments, the treatment method may be used to ameliorate a condition comprising aqueous tear-deficient dry eye syndrome, evaporative dry eye syndrome, blepharitis (i.e., eyelid inflammation), keratoconjunctivitis sicca, eye redness, corneal ulcer due to dryness, or any combination thereof. In such embodiments, the treatment method may be further defined as a method of reducing ocular dryness in the subject, reducing ocular inflammation in the subject, enhancing tear layer formation in the subject, improving or enhancing a quality of life of the subject, or combinations thereof. However, it is to be appreciated that the effect(s) of the composition may also be used preventatively, as described above, and the treatment method may thus be further defined as a method of maintaining or managing eye heath or function in the subject, preventing ocular inflammation in the subject, preventing dry eye in the subject, or combinations thereof.

As will be understood in view of the Examples and description here, the treatment method may be used to ameliorate such a condition via PPARγ activation, anti-inflammatory, and/or antioxidant effects. More specifically, the treatment method may be used to ameliorate a condition via increasing lipid synthesis in a Meibomian gland of the subject, increase meibum production of the subject, increase meibum secretion of the subject, or combinations thereof. In these or other embodiments, the treatment method is used to ameliorate a condition by inhibiting or reducing the activity of inflammation-associated transcription factors and associated enzymes, such as via inhibition of NF-κB. In particular, without being bound or limited to any particular theory, it is believed that PPARγ has an anti-inflammatory role and plays a role in Meibomian gland function. PPARγ-active botanical extracts (i.e., botanical extracts exhibiting upregulating or activating PPARγ) may improve the function of Meibomian glands and their lipid synthesis and secretion, which in turn may improve the outer layer of human tear film to prevent evaporation of the aqueous tear film component associated with dry eye and dry eye-associated ocular conditions. Furthermore, inflammation is believed to be a core mechanism of dry eye disease, and can be triggered by extrinsic and intrinsic factors alike. Once initiated, the inflammatory response can become cyclical, leading to enhanced discomfort. As NF-κB can stimulate the production of inflammatory cytokines, it is also believed that inhibition of NF-κB activity provides a beneficial effect for eye health and in the amelioration, treatment, and/or prevention of dry eye.

The following examples, illustrating the compositions and methods of this disclosure, are intended to illustrate and not to limit the disclosure.

### EXAMPLES

Various materials, components, and reagents utilized in the Examples are set forth in Table 1 below.

TABLE 1
Abbreviations

| Abbreviation | Definition/Description |
|---|---|
| PPARγ | Peroxisome proliferator-activated receptor gamma |
| GAL4/UAS | GAL4 is the yeast gene encoding the yeast protein, |
| | Gal4; Upstream Activation Sequence to which Gal4 |
| | binds |
| CHO-K1 | Chinese hamster ovary cells |
| HMGEC | Immortalized male human meibomian gland epithelial |
| | cells |
| Rosi | Rosiglitazone |
| DMEM/F12 | Gibco Dulbecco's Modified Eagle Medium: Nutrient |
| | Mixture F-12, available from Thermo Fisher |
| | Scientific |
| EGF | Epidermal growth factor |
| DMSO | Dimethyl sulfoxide, having chemical formula |
| | (CH₃)₂SO |
| Media 1 | DMEM/F12 containing EGF (10 ng/mL) and albumin |
| | (600 μg/mL) |
| LipidTox | HCS LipidTOX ™ Green Phospholipidosis Detection |
| | Reagent, available from ThermoFisher (Waltham, MA) |
| qPCR | Quantitative (or real-time) polymerase chain reaction |
| ADFP | Adipose differentiation-related protein, (i.e., |
| | perilipin 2, adipophilin) |
| ANGPTL4 | Angiopoietin-like 4 |
| ELOVL4 | Elongation of very long chain fatty acids-like 4 |
| T0070907 | Blocker of PPARγ transcriptional activity, available |
| | from Tocris (Minneapolis, MN) |
| FBS | Fetal bovine serum |
| G418 | Geneticin, aminoglycoside antibiotic |
| Loquat | Eriobotrya japonica extract (Loquat Leaf extract, |
| Extract #1 | 10%; AuNutra Industries, Chino, CA) |
| Loquat | Eriobotrya japonica extract (Loquat Leaf extract, |
| Extract #2 | 5%; AuNutra Industries, Chino, CA) |
| Ginger | Zingiber officinale extract (Verdure Sciences, |
| Extract #1 | Noblesville, IN). |
| Ginger | Zingiber officinale root extract (Select Botanical, |
| Extract #2 | Barcelona, Spain) |

### Example 1: GAL4/UAS/PPARγ Luciferase Reporter Assay

A library of botanical extracts is screened for PPARγ ligand binding domain (LBD) activity using a GAL4/UAS luciferase reporter assay. More specifically, a PPARγ LBD is PCR amplified from full-length human PPARγ gene (MGC 5041, Open Biosystems, Cambridge, UK) and ligated into a modified pFN26A (BIND) vector (Promega, Madison, Wis.) to give a Gal4-PPARγ-LBD fusion protein construct. Chinese hamster ovary cells (CHO-K1) (ATCC, Manasas, Va.) are grown in F12K growth media (ThermoFisher, Waltham Mass.) supplemented with 10% FBS, penicillin/streptomycin, and amphotericin B in an incubator (37° C.; 5% CO2). The CHO-K1 cells are stably transfected with a pGL4.35 vector (Promega, Madison, Wis.) using Fugene 6 (Promega). Monoclonal cell lines are selected under Hygromycin treatment, and surviving clones stably transfected with the Gal4-PPARγ-LBD fusion protein construct with selection of stable cell lines from G418-supplemented media treated cells. Dually transfected stable cell lines are maintained in F12K growth media supplemented with hygromycin and G418.

To test botanical extracts for PPARγ LBD activity, the above referenced cell line is plated at 3.0×10⁴ cells/well in white-walled, clear bottom, 96-well plates and incubated overnight, and subsequently serum-starved for 24 hours. Botanical extracts are prepared as 100 mg/mL solutions in 100% DMSO, vortexed and sonicated for 0.5 hours. Extracts are serially diluted with 100% DMSO and then diluted to 10× final concentrations with serum-free F12K medium. Final test concentrations start at 100 μg/mL with treatment times of approximately 18 hours. Treatment with troglitazone (Sigma Aldrich, St. Louis, Mo.) in select wells is performed as a control to verify assay activity. Following treatment, the media is aspirated and cells are lysed using Biotium lysis buffer (Fremont, Calif.), followed by addition of a d-luciferin substrate (Biotium). Luminescence is then measured using the SpectraMax M5 spectrophotometer (Molecular Devices, Sunnyvale, Calif.) and the results compared to vehicle control (DMSO-supplemented F12K medium) treated cell responses. Four botanical extracts exhibiting positive PPARγ LBD activity are identified, and shown in Table 2 below.

TABLE 2
PPARγ LBD Active Botanical Extracts

| | Activity |
|---|---|
| Sample | (% vehicle control) |
| Loquat Extract #1 | 273 |
| Loquat Extract #2 | 438 |
| Ginger Extract # 1 | 131 |
| Ginger Extract # 2 | 265 |

The four extracts are further evaluated for PPARγ-mediated lipid synthesis activity, as described below.

### Example 2: PPARγ-Mediated Lipid Synthesis Activity

Extracts of loquat and ginger identified in Example 1 are tested for their effect on lipid synthesis and PPARγ receptor activation using HMGECs. In particular, HMGECs are plated on collagen coated glass cover slips and treated with samples of each extract (varying concentrations in Media 1 with 0.1% DMSO), positive control (Rosi, 30 μM in Media 1 with 0.1% DMSO), and negative control (Media 1 with 0.1% DMSO) and incubated for 6 days. The treated cells are then fixed, stained with LipidTox Green, and the area of neutral lipid staining quantified. The results of the quantification are shown in Tables 3-6 below, with data expressed as percent positive control, in which the relative fluorescence units elicited by positive control cells exposed only to Rosi is set at 100%.

TABLE 3
PPARγ-Mediated Lipid Synthesis Activity of Loquat Extract # 1

| Treatment | Nuclei | | Lipid | | % Lipid | |
|---|---|---|---|---|---|---|
| Sample (Amount) | Mean | SD | Mean | SD | Mean | SD |
| Vehicle | 1858.67 | 117.23 | 0.252562 | 0.286609 | 0.0% | 7.2% |
| Rosi (30 μM) | 1622.13 | 266.868 | 4.206128 | 1.046069 | 97.5% | 25.9% |
| Loquat Extract # 1 (12 mg) | 1196 | 373.367 | 35.78703 | 24.46309 | 979.5% | 627.4% |
| Loquat Extract # 1 (25 mg) | 979.125 | 240.305 | 50.96758 | 5.517507 | 1289.3% | 132.0% |
| Loquat Extract # 1 (50 mg) | 583.286 | 204.055 | 62.55341 | 13.94998 | 1575.8% | 352.8% |

TABLE 4
PPARγ-Mediated Lipid Synthesis Activity of Loguat Extract # 2

| Treatment | | | Nuclei | | Lipid | | % Lipid | |
|---|---|---|---|---|---|---|---|---|
| Sample | Amount | | Mean | SD | Mean | SD | Mean | SD |
| Vehicle | — | | 1432 | 411.7 | 0.445 | 0.351 | 0.0% | 1.7% |
| Rosi | 30 | μM | 1070 | 377.4 | 21.26 | 6.82 | 100.0% | 32.8% |
| Loquat Extract # 2 | 12 | mg | 862.7 | 289 | 8.321 | 2.626 | 38.6% | 13.3% |
| Loquat Extract # 2 | 25 | mg | 701.9 | 163.3 | 18.17 | 5.688 | 85.1% | 27.3% |
| Loquat Extract # 2 | 50 | mg | 1061 | 353.8 | 19.21 | 7.513 | 90.2% | 36.1% |

TABLE 5
PPARy-Mediated Lipid Synthesis Activity of Ginger Extract # 1

| Treatment | | | Lipid | | % Lipid | |
|---|---|---|---|---|---|---|
| Sample | Amount | | Mean | SD | Mean | SD |
| Vehicle | — | | 0.393 | 0.418 | 0% | 26% |
| Rosi | 30 | μM | 1.979 | 1.108 | 100% | 70% |
| Ginger Extract # 1 | 12 | mg | 4.504 | 2.782 | 259% | 175% |
| Ginger Extract # 1 | 25 | mg | 3.525 | 2.902 | 198% | 183% |

TABLE 6
PPARγ-Mediated Lipid Synthesis Activity of Ginger Extract # 2

| Treatment | | | Nuclei | | Lipid | | % Lipid | |
|---|---|---|---|---|---|---|---|---|
| Sample | Amount | | Mean | SD | Mean | SD | Mean | SD |
| Vehicle | — | | 1432 | 411.7 | 0.445 | 0.351 | 0.0% | 1.7% |
| Rosi | 30 | μM | 1070 | 377.4 | 21.26 | 6.82 | 100.0% | 32.8% |
| Ginger Extract # 2 | 12 | mg | 951.9 | 221 | 6.549 | 2.789 | 29.3% | 13.4% |
| Ginger Extract # 2 | 25 | mg | 784.7 | 137.6 | 7.426 | 2.67 | 33.5% | 12.8% |
| Ginger Extract # 2 | 50 | mg | 828.1 | 239.4 | 6.239 | 2.357 | 27.8% | 11.3% |

As shown, the botanical extracts of Loquat Leaf and Ginger give increased lipid synthesis at amounts equal to or greater than those obtained with Rosi.

### Example 3: Upregulation of PPARγ Response Gene ADFP, ELOVL4, ANGPTL4, & PPAγ

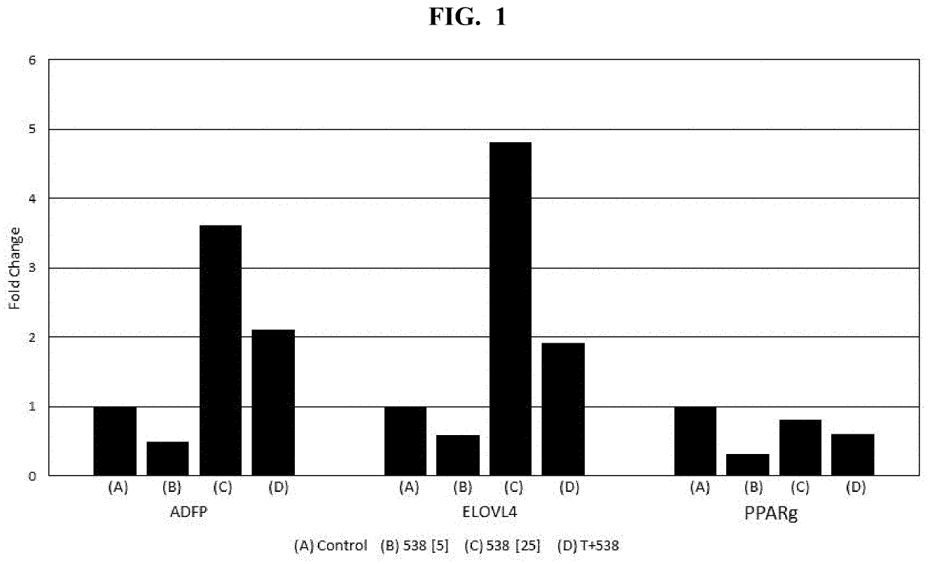

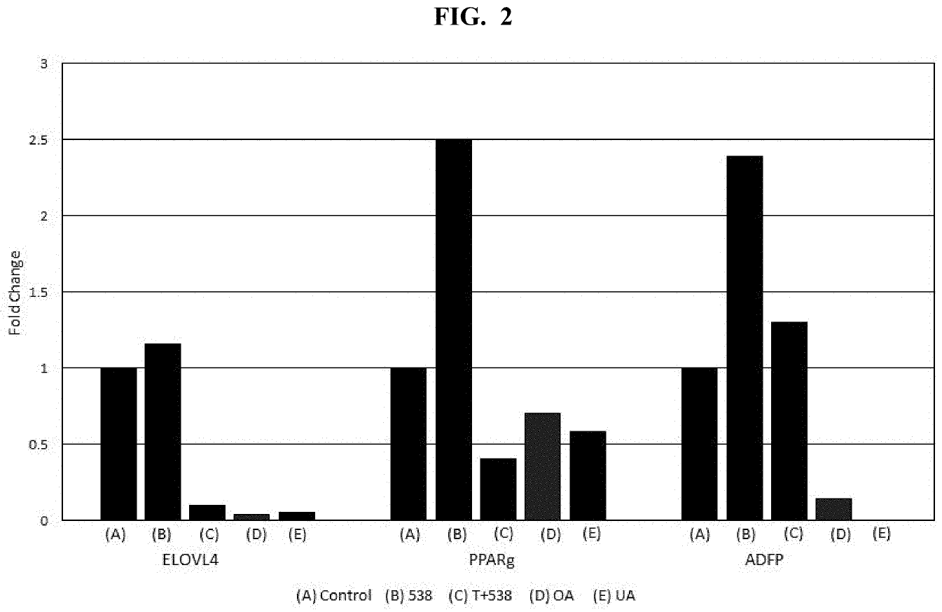

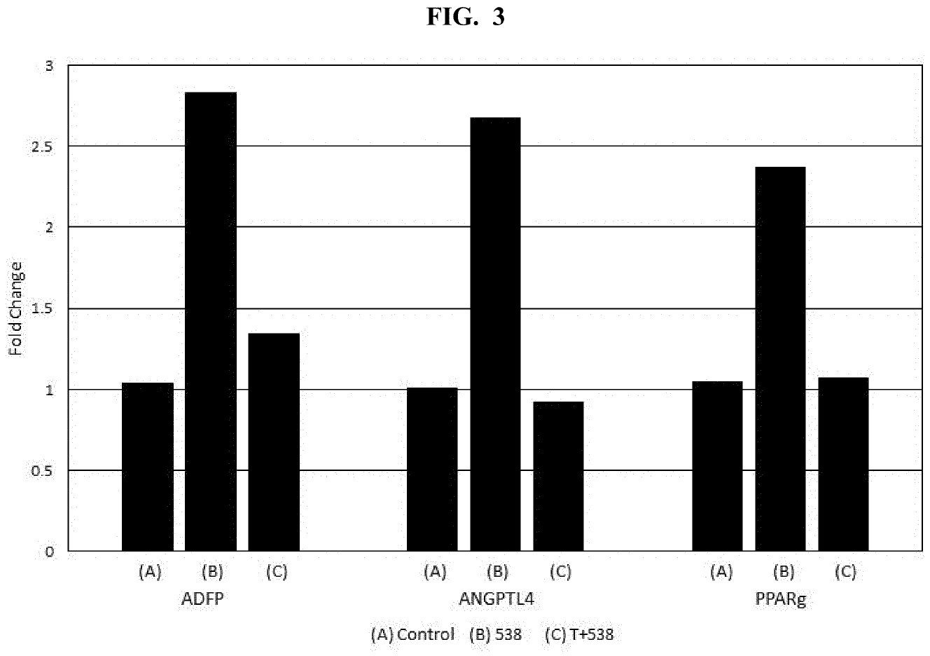

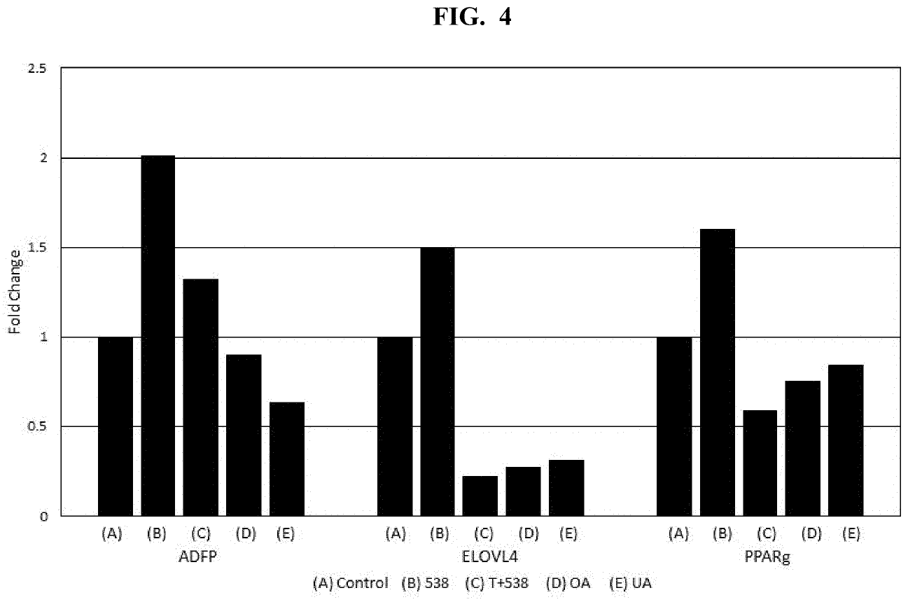

Loquat Extract #1 (“538”), and known phytochemical components thereof (oleanolic acid (“OA”); ursolic acid (“UA”)), are analyzed by real-time PCR (BioRad CFX96 Thermal Cycler, Hercules, Calif. (or equivalent)), to assess sample-induced expression of the PPARγ response genes ADFP, ELOVL4, and ANGPTL4 via mRNA quantification. In particular, samples are prepared and analyzed according to the procedure set forth in Jester et. al., “PPARγ regulates mouse meibocyte differentiation and lipid synthesis,” The Ocular Surface 14.4 (2016): 484-494, the disclosure of which is herein incorporated by relevance. The results of the quantification are shown in FIGS. 1-4, where the vertical (Y) axis of each plot corresponds to the fold change in response observed.

As shown, Loquat Extract #1 (“538”) increases mRNA of ADFP, ELOVL4, ANGPTL4 in a dose-dependent manner. To assess the effects on gene expression mediation via PPARγ signaling, a PPARγ-specific antagonist (T0070907; (“T”)) is added to inhibit PPARγ signaling. As shown, the PPARγ response gene upregulatory effects of Loquat Extract #1 is at least partly suppressed by inhibiting PPARγ signaling, evidencing that the above measured effects on gene expression are mediated through PPARγ signaling. In particular, Loquat leaf extract, the most potent inducer identified (1575% of Rosi), consistently increases gene expression of ADFP, ELOVL4, ANGPTL4, and PPARγ, with an effect blocked by a PPARγ antagonist. Certain known phytochemical components of Loquat leaf (i.e., oleanolic acid (“OA”); ursolic acid (“UA”)) showed no individual effects on lipid synthesis or PPARγ response gene expression. As such, Loquat leaf is shown to have specific and synergistic PPARγ agonist activity that may be unrelated to individual phytochemical components thereof.

The terms “comprising” or “comprise” are used herein in their broadest sense to mean and encompass the notions of “including,” “include,” “consist(ing) essentially of,” and “consist(ing) of.” The use of “for example,” “e.g.,” “such as,” and “including” to list illustrative examples does not limit to only the listed examples. Thus, “for example” or “such as” means “for example, but not limited to” or “such as, but not limited to” and encompasses other similar or equivalent examples. The term “about” as used herein serves to reasonably encompass or describe minor variations in numerical values measured by instrumental analysis or as a result of sample handling. Such minor variations may be in the order of ±0-10, ±0-5, or ±0-2.5, % of the numerical values. Further, the term “about” applies to both numerical values when associated with a range of values. Moreover, the term “about” may apply to numerical values even when not explicitly stated.

Generally, as used herein a hyphen “-” or dash “-” in a range of values is “to” or “through”; a “>” is “above” or “greater-than”; a “≥” is “at least” or “greater-than or equal to”; a “<” is “below” or “less-than”; and a “≤” is “at most” or “less-than or equal to.” On an individual basis, each of the aforementioned applications for patent, patents, and/or patent application publications, is expressly incorporated herein by reference in its entirety in one or more non-limiting embodiments.

It is to be understood that the appended claims are not limited to express and particular compounds, compositions, or methods described in the detailed description, which may vary between particular embodiments which fall within the scope of the appended claims. With respect to any Markush groups relied upon herein for describing particular features or aspects of various embodiments, it is to be appreciated that different, special, and/or unexpected results may be obtained from each member of the respective Markush group independent from all other Markush members. Each member of a Markush group may be relied upon individually and or in combination and provides adequate support for specific embodiments within the scope of the appended claims.

It is also to be understood that any ranges and subranges relied upon in describing various embodiments of the present invention independently and collectively fall within the scope of the appended claims, and are understood to describe and contemplate all ranges including whole and/or fractional values therein, even if such values are not expressly written herein. One of skill in the art readily recognizes that the enumerated ranges and subranges sufficiently describe and enable various embodiments of the present invention, and such ranges and subranges may be further delineated into relevant halves, thirds, quarters, fifths, and so on. As just one example, a range “of from 0.1 to 0.9” may be further delineated into a lower third, i.e., from 0.1 to 0.3, a middle third, i.e., from 0.4 to 0.6, and an upper third, i.e., from 0.7 to 0.9, which individually and collectively are within the scope of the appended claims, and may be relied upon individually and/or collectively and provide adequate support for specific embodiments within the scope of the appended claims. In addition, with respect to the language which defines or modifies a range, such as “at least,” “greater than,” “less than,” “no more than,” and the like, it is to be understood that such language includes subranges and/or an upper or lower limit. As another example, a range of “at least 10” inherently includes a subrange of from at least 10 to 35, a subrange of from at least 10 to 25, a subrange of from 25 to 35, and so on, and each subrange may be relied upon individually and/or collectively and provides adequate support for specific embodiments within the scope of the appended claims. Finally, an individual number within a disclosed range may be relied upon and provides adequate support for specific embodiments within the scope of the appended claims. For example, a range “of from 1 to 9” includes various individual integers, such as 3, as well as individual numbers including a decimal point (or fraction), such as 4.1, which may be relied upon and provide adequate support for specific embodiments within the scope of the appended claims.

The present invention has been described herein in an illustrative manner, and it is to be understood that the terminology which has been used is intended to be in the nature of words of description rather than of limitation. Many modifications and variations of the present invention are possible in light of the above teachings. The present invention may be practiced otherwise than as specifically described within the scope of the appended claims. The subject matter of all combinations of independent and dependent claims, both single and multiple dependent, is herein expressly contemplated.

What is claimed is:

1. A composition for administration to a subject with dry eye or a dry eye-associated ocular condition, the composition comprising a botanical active agent comprising a Zingiber officinale extract, an Eriobotrya japonica extract, or a combination thereof, the botanical active agent being present in the composition in an amount effective for ameliorating dry eye or a dry eye-associated ocular condition in a subject upon administration of the composition thereto.

2. The composition of claim 1, wherein the botanical active agent is present in the composition in an amount effective to, upon administration of the composition to a subject: (i) increase lipid synthesis in a Meibomian gland of the subject; (ii) increase meibum production of the subject; (iii) increase meibum secretion of the subject; or (iv) any combination of (i)-(iii).

3. The composition of claim 1, wherein the botanical active agent is present in the composition in an amount effective to, upon administration of the composition to a subject: (i) increase expression of messenger ribonucleic acid (mRNA) for adipose differentiation-related protein (ADFP) in the subject; (ii) increase expression of mRNA for very long chain fatty acids-like 4 (ELOVL4) in the subject; (iii) increase expression of mRNA for angiopoietin-like 4 (ANGPTL4) in the subject; or (iv) any combination of (i)-(iii).

4. The composition of claim 1, wherein the botanical active agent is present in an amount effective to activate peroxisome proliferator-activated receptor gamma (PPARγ) in Meibomian glands of a subject upon administration of the composition thereto.

5. The composition of claim 1, wherein the botanical active agent is present in an amount effective to inhibit nuclear factor kappa-light-chain-enhancer of activated B cells (NF-κβ) in cells of a subject upon administration of the composition thereto.

6. The composition of claim 1, wherein the botanical active agent is present in an amount of from 1 to 2000 mg per dose of the composition.

7. The composition of claim 1, wherein the botanical active agent comprises: (i) from 1 to 1000 mg of the Zingiber officinale extract per dose of the composition; (ii) from 1 to 1000 mg of the Eriobotrya japonica extract per dose of the composition; or (iii) both (i) and (ii).

8. The composition of claim 1, wherein the composition further comprises an eye health additive component comprising: (i) lutein; (ii) beta carotene; (iii) lycopene; (iv) alpha carotene;
(v) zeaxanthin; (vi) docosahexaenoic acid (DHA); (vii) eicosapentaenoic acid (EPA); (viii) vitamin C; (ix) vitamin A; (x) a bilberry extract; (xi) a citrus bioflavonoid extract; (xii) a black currant extract; (xiii) a spinach dehydrate; or (xiv) any combination of (i)-(xiii).

9. The composition of claim 8, wherein the eye health additive component is present in the composition in an amount of from 1 to 1000 mg per dose of the composition.

10. The composition of claim 1, further comprising a pharmaceutically acceptable additive.

11. The composition of claim 1, wherein the composition is formulated for oral administration to the subject.

12. The composition of claim 11, wherein the composition is adapted to be: (i) consumed as a liquid; (ii) consumed as a dry powder; (iii) mixed with a foodstuff; or (iv) any combination of (i)-(iii).

13. The composition of claim 11, wherein the composition comprises a softgel or capsule shell encapsulating the botanical active agent.

14. A method of ameliorating dry eye or a dry eye-associated ocular condition, said method comprising administering an effective amount of a composition comprising a botanical active agent to a subject in need thereof, wherein the composition is according to claim 1.

15. The method of claim 14, wherein the dry eye-associated ocular condition comprises: (i) aqueous tear-deficient dry eye syndrome; (ii) evaporative dry eye syndrome; (iii) blepharitis (eyelid inflammation); (iv) keratoconjunctivitis sicca; (v) eye redness; (vi) corneal ulcer due to dryness; or (vii) any combination of (i)-(vi).

16. The method of claim 14, wherein the composition is administered to the subject in an amount effective to: (i) increase lipid synthesis in a Meibomian gland of the subject; (ii) increase meibum production of the subject; (iii) increase meibum secretion of the subject; or (iv) any combination of (i)-(iii).

17. The method of claim 14, wherein the composition is administered to the subject in a dosage formulation configured to provide to the subject: (i) from 1 to 2000 mg of the botanical active agent per dose; (ii) from 1 to 1000 mg of the Zingiber officinale extract per dose; (iii) from 1 to 1000 mg of the Eriobotrya japonica extract per dose; or (iv) any combination of (i)-(iii).

18. The method of claim 14, wherein the method further comprises administering an eye health additive to the subject, the eye health additive comprising: (i) lutein; (ii) beta carotene; (iii) lycopene; (iv) alpha carotene; (v) zeaxanthin; (vi) docosahexaenoic acid (DHA); (vii) eicosapentaenoic acid (EPA); (viii) vitamin C; (ix) vitamin A; (x) a bilberry extract; (xi) a citrus bioflavonoid extract; (xii) a black currant extract; (xiii) a spinach dehydrate; or (xiv) any combination of (i)-(xiii).

19. The method of claim 14, wherein the composition is administered orally to the subject.

20. The method of claim 19, wherein the composition comprises a softgel or capsule shell encapsulating the botanical active agent.
